(19) United States
(12) Patent Application Publication
(10) Pub. No.: US 2022/0389357 A1
(43) Pub. Date: Dec. 8, 2022

(54) METHOD AND DEVICE FOR THE PRODUCTION OF MICROALGAE BIOMASS

(71) Applicant: IGV Institut fuer Getreideverarbeitung GmbH, Nuthetal (DE)

(72) Inventors: Gerd Huschek, Schildow (DE); Horst Theisen, Bonn (DE)

(21) Appl. No.: 17/774,923

(22) PCT Filed: Nov. 4, 2020
(86) PCT No.: PCT/DE2020/100939

§ 371 Date: May 6, 2022

(30) Foreign Application Priority Data
Nov. 7, 2019 (DE) 10 2019 130 109.2

(51) Int. Cl. C12M 1/00; C12M 1/12; C12M 1/26; C12M 1/34

(52) U.S. Cl. CPC C12M 21/02; C12M 23/22; C12M 25/01; C12M 29/06; C12M 29/18; C12M 31/02; C12M 31/10; C12M 33/10; C12M 41/06; C12M 41/26; C12M 41/28; C12M 41/36

(57) ABSTRACT

The invention relates to the production of microalgae biomass. The microalgae contained in a suspension of water and microalgae are continuously phototrophically or mixotrophically cultivated in a cultivation module (1), which is passed multiple times by the suspension and has a gas part and a liquid part with a liquid supply (3), by supplying light from at least one artificial light source (5) and nutrients. According to the turbidity established by sensors, volume fractions of the suspension are repeatedly discharged from the cultivation module (1) for the harvest of microalgae and removed by means of a centrifuge (7). The cultivation of the microalgae occurs in an climate chamber forming the cultivation module (1), which is operated using water. Alongside a regulating of the temperature of the suspension, there also occurs a regulating of its pH value via the controlled addition of buffer ions and a regulating of the redox potential of the suspension and thereby also of its microbial contamination by controlling the light and nutrient supply, as well of a metered addition of oxygen. In addition, after the removal of microalgae, the remaining suspension is irradiated with UV light in order to kill unwanted microbial contamination before being returned into the cultivation module (1).

The invention relates to a solution for production of microalgae biomass that particularly advantageously allows large-scale production, namely the production of very large quantities of microalgae biomass on an industrial scale. More particularly, the solution presented allows season-independent large-scale production of microalgae biomass with a simultaneously comparatively low space requirement. The subjects of the invention are a corresponding process and an installation, suitable for carrying out said process, for production of microalgae biomass.

A growing demand for microalgae biomass has been observed for several years and can also still be observed at present. Microalgae, i.e., the biomass thereof which may be enriched with selected secondary ingredients such as, for example, vitamins or omega-3 fatty acids or minerals or bioactive proteins/peptides, is, inter alia, used in foods, in food supplements, for medical purposes and in cosmetics in a wide variety of forms.

In order to be able to cover the existing demand for microalgae biomass, ever larger installations for production of large quantities of microalgae biomass have been constructed more recently. The microalgae providing the biomass are phototrophically or mixotrophically cultivated in a suspension consisting of water and the particular microalgae. The cultivation of the microalgae is carried out with addition of nutrients, with or without further substances to be enriched in the microalgae, to the suspension by subjecting the suspension over relatively long periods of time to exposure to light, which is absolutely necessary for the growth of the microalgae, and to supply of nutrients, such as, in particular, CO₂ as gaseous nutrient. In order to allow a relatively long exposure of the suspension to light and nutrients (especially CO₂), the suspension, in processes customary for this purpose, is usually guided in a photobioreactor along relatively long paths or in a circuit which is passed through multiple times.

When the microalgae have proliferated to a sufficient extent owing to sustained exposure to light and nutrients, the suspension or at least portions thereof is/are generally centrifuged in a centrifuge to separate the microalgae contained in the suspension from the liquid constituents. The microalgae centrifuged out are then generally subjected to a drying process and lastly further processed according to whatever their intended purpose is.

Different concepts have been developed for the cultivation of microalgae, i.e., their exposure to light and nutrients over a period of time. For example, for this purpose, use is made of so-called tubular bioreactors, in which a suspension containing the particular microalgae is guided along relatively long paths in an arrangement of transparent tubes, namely generally glass tubes, and exposed at the same time to the light with simultaneous introduction of nutrients into the tubes. Daylight is frequently used for exposure of the microalgae or the suspension containing said microalgae to light, with assistance by artificial light sources as well if necessary. Such a tubular bioreactor is, for example, known from DE 10 2009 028 474 A1.

Another concept is to use suitable nozzles to finely spray a suspension containing the microalgae in a chamber flooded with light and supplied with CO₂ as gaseous nutrient. The droplets of the suspension finely sprayed in the upper region of such a chamber gradually float to the bottom in the chamber, said droplets, or the microalgae contained therein, being exposed to the light in the chamber for the duration of this downward movement. At the bottom of the chamber, the suspension is collected in the form of the droplets gathering there and then supplied back to its nozzles, which are used for spraying in the aforementioned chamber, by means of a piping and pump system.

After some time and multiple passage through the circuit explained above, which is associated with corresponding growth and proliferation of the microalgae contained in the circulating suspension, the microalgae is in turn harvested by centrifugation out of the suspension.

A process using the last-explained principle and a photobioreactor operating in accordance with said process are, for example, described in EP 2 446 016 B1. US 2011/0312062 A1, too, discloses a photobioreactor which uses said principle in accordance with a possible embodiment.

Moreover, it is known from document EP 2 446 016 B1, which deals generally with the phototrophic or mixotrophic cultivation of organisms or cells, to arrange specific structures in the chamber, in the upper region of which the suspension is applied by spraying, which structures make it possible to specifically retard the gravitational downward movement of the droplets in the chamber, so that the microalgae contained in the droplets are exposed to the light and to the nutrients introduced into the chamber, such as CO₂ in particular, for longer while they are in the chamber. The document also addresses the possibility of optionally artificially generating the light to which the microalgae are exposed.

Regardless of whichever principle is used, installations for cultivation of microalgae by means of bioreactors of the forms described above typically have a very large space requirement—at least when they are used for generation of relatively large quantities of biomass—or are, as in the case of the embodiment described in DE 10 2009 028 474 A1, only designed for the production of relatively small quantities of microalgae biomass. Moreover, bioreactors are usually at least predominantly operated using daylight or natural sunlight. Therefore, appropriate installations for generation of relatively large quantities of microalgae biomass are very frequently also constructed in regions which are conveniently located due to the climate and are sparsely populated and therefore have large spaces available. However, this does not change the fact that appropriate installations have low space-efficiency and are subjected to seasonal fluctuations in whatever natural light is available.

It is an object of the invention to provide a solution which allows large-scale production of microalgae biomass that is season-independent and highly space-efficient. A process and an installation, suitable for carrying out the process, for production of microalgae biomass are to be specified to this end.

The object is achieved by a process having the features of claim 1. An installation for production of microalgae biomass that achieves the object and is suitable for carrying out the process is characterized by the first independent product claim. Advantageous embodiments and developments of the invention are given by the respective dependent claims.

The proposed process for producing microalgae biomass is based on an approach in which microalgae contained in a suspension of water and microalgae are phototrophically or mixotrophically cultivated in a continuous circulation in a cultivation module through which the suspension passes multiple times with supply of light and of nutrients, including CO₂ as gaseous nutrient. However, only light from at least one artificial light source is used for the process described in more detail below.

Volume fractions of the circulating suspension are repeatedly discharged from the cultivation module for harvesting of microalgae and centrifuged by means of a centrifuge. The volume fractions of the suspension not discharged for harvesting of the microalgae remain in the cultivation module formed by a gas portion and a liquid portion having a liquid reservoir, in that they are resupplied from the liquid reservoir to the gas portion of the cultivation module, where they are applied by atomization. Volume fractions of the circulating suspension are discharged from the cultivation module for the purpose of harvesting of microalgae whenever the turbidity of the suspension, as established by means of optical sensors, exceeds a minimum value.

However, the minimum turbidity value to be set in this respect cannot be firmly specified. Not least, said minimum value should undoubtedly also depend on the species of whichever microalgae are to be cultivated. However, it should not be set too low, since otherwise only a comparatively small quantity of microalgae can be centrifuged out of whatever volume fractions of the suspension are discharged, and the harvesting process may then be less efficient. Ultimately, however, this is basically an optimization task in connection with the configuration and adjustment of an appropriate installation with respect to the cultivation of a specific species of microalgae.

In the process proposed to achieve the object, the microalgae are cultivated in a cultivation module designed as a climatic chamber. The aforementioned climatic chamber is operated in a water-economical manner. This water-economical mode of operation involves not only regulating the temperature of the suspension, but also regulating the pH thereof by means of the controlled addition of buffer ions and regulating the redox potential of the suspension and hence the microbial contamination thereof by means of control of the supply of light and nutrients and of the metered addition of oxygen.

Furthermore, according to the invention, the suspension having a high proportion of water and an only very low proportion of microalgae, which suspension remains after centrifugation of microalgae out of the discharged volume fractions, is recycled into the cultivation module, said suspension being irradiated, before it is recycled into the cultivation module, with UV light to kill undesirable microbial contamination until a minimum redox potential measured in said volume fractions of the suspension is reached. The residence time of the suspension to be recycled into the cultivation module in the region of a UV light source used for the irradiation thereof is inversely proportional to whatever redox potential is established for the suspension in a repeated measurement. Thus, the higher the redox potential, the shorter the time for which the suspension (predominantly only water after centrifugation) must still be exposed to the UV light.

In one embodiment of the process according to the invention presented in principle above, what can take place is sequence, according to which the following process steps are passed through multiple times:
A. Spraying volume fractions of the suspension, which volume fractions contain microalgae to be cultivated and are taken from the liquid reservoir, in the upper region of the gas portion of the cultivation module into which nutrients, including at least CO₂ as gaseous nutrient, are introduced.
B. Retarding the gravitational downward movement of droplets of the suspension that arise upon spraying and contain the microalgae, by means of suitable structural elements arranged for this purpose in the gas portion of the cultivation module. Further details about the nature of the aforementioned structural elements shall be provided in connection with the explanation of the proposed installation suitable for carrying out the process according to the invention.
C. Exposing the microalgae contained in the droplets retarded in their downward movement to the light from LED light sources arranged in the gas portion of the cultivation module, which light is tailored to the species of the microalgae to be cultivated with respect to the wavelength thereof and the intensity thereof, and to the CO₂ introduced into the cultivation module. The intensity of the light emitted by the LED light sources changes as the cultivation process progresses, namely increases with increasing turbidity of the suspension.
D. Recycling downwardly moving, microalgae-containing droplets into the liquid reservoir formed at the bottom or in a container arranged below the gas portion of the cultivation module after passage through the gas portion of the cultivation module.
E. Discharging volume fractions of suspension recycled for respraying in the gas portion of the cultivation module from the liquid reservoir and harvesting microalgae contained in the discharged volume fractions by centrifugation in a centrifuge. Appropriate volume fractions of the suspension are discharged for the purpose of harvesting microalgae if the turbidity of the volume fractions of the suspension that are supplied back to the gas portion of the cultivation module for respraying as per the first process step (process step A.), which turbidity is established by means of optical sensors, exceeds a minimum value. Besides the volume fractions of the suspension that are resupplied to the gas portion of the cultivation module, the suspension remaining after centrifugation of the discharged volume fractions is also recycled back into the gas portion of the cultivation module, these volume fractions of the suspension being irradiated with UV light beforehand.

In connection with the process proceeding according to the process steps explained above, the pH of the suspension in the liquid portion of the cultivation module is more or less constantly measured and regulated by controlled metered addition of buffer ions. The pH is kept in a range between 7 and 8, preferably between 7 and 7.5, which promotes the growth of the microalgae. Moreover, microbial contamination of the suspension is regulated through monitoring of the redox potential thereof, which monitoring is done by period measurement in the liquid portion of the cultivation module (climatic chamber). The regulation is done by increasing the supply of nutrients and the light intensity of the at least one LED light source if the redox potential exceeds an upper limit and reducing the supply of nutrients and the light intensity of the at least one LED light source and also increasing the metered addition of oxygen that takes place in the liquid portion of the cultivation module if the redox potential falls below a lower limit.

At the same time, as already stated at the start, the light intensity of the LED light sources is additionally controlled depending on the turbidity of the suspension, which turbidity is determined as per the 5th process step (process step E.), such that the light intensity is adjusted proportionally to the turbidity.

Insofar as repeated reference is made above and in the claims to volume fractions of the suspension in connection with the discharging of suspension from the circulation (circuit) or from the liquid portion of the cultivation module and with the resupplying of the suspension to the gas portion of the cultivation module, this is intended to express that it is only a portion of the suspension, not the entire suspension, that is concerned in each case.

How large the particular volume fraction of the suspension preferably is or should meaningfully be, especially whatever volume fraction of the suspension is discharged from the liquid portion of the cultivation module for harvesting of the microalgae, would have to be defined in connection with implementing the process as a result of appropriate test runs. From calculations and simulations and as a result of tests carried out on a laboratory scale, it has become apparent that it might be advantageous to discharge a volume fraction of 15% to 50% of the volume of the suspension present in the liquid portion of the cultivation module.

Moreover, it can be seen from the explanations above and from the presence of a reservoir that the entire quantity of the suspension is never simultaneously present in the circulation of the circuit system formed by the gas portion and the liquid portion of the cultivation module. On the contrary, certain volume fractions of the suspension are present in the liquid reservoir at least temporarily and are therefore in a sense (not biologically) at rest at least briefly.

To regulate the pH of the suspension, i.e., to set a pH of preferably 7 to 7.5, what can be effected, for example, is a metered addition of calcium and/or magnesium ions as buffer ions, said ions being metered into the liquid portion of the cultivation module. With regard to the question of dividing the climatic chamber into a gas portion and a liquid portion, a few details shall be provided in connection with the description of the installations suitable for carrying out the process.

According to an advantageous development of the process, the surface temperature of the LED light sources arranged in the cultivation module can be used for temperature control of the suspension, and said surface temperature can in turn be regulated by reducing or increasing the volumetric flow rate of a cooling medium used for cooling of the LED light sources. Besides the CO₂ already repeatedly mentioned as gaseous nutrient, nitrogen (e.g., in the form of ammonia) and/or phosphorus and/or carbon (e.g., from carbon sources such as glucose) are typically additionally supplied as nutrients to the suspension in the liquid portion of the cultivation module.

Furthermore, the process can be conducted such that specific secondary ingredients are specifically formed or enriched in the microalgae. To this end, substances or groups of substances from one or more of the following can be supplied in a controlled quantity to the suspension in the liquid portion of the cultivation module, taking into account appropriate stress factors during cultivation:
nutrients and microbiological contamination for formation of vitamins,
nutrients for formation of omega-3 fatty acids,
nutrients for formation of bioactive proteins/peptides,
minerals to be bound by the microalgae,
zinc or iron to be bound by the microalgae.

The process according to the invention allows highly space-efficient production of large quantities of algae biomass. The use of a climatic chamber as a cultivation module, operated in a water-economical manner, achieves a very strong and robust growth of the microalgae and allows the generation of large quantities of microalgae biomass of a very high and consistent quality especially from the viewpoint of possible microbial contamination, the process regime also outstandingly making it possible with its pin-point adjustability of essential process parameters to enrich the biomass with selected secondary substances, such as vitamins and/or minerals, in a precisely defined concentration.

The process makes it possible to produce microalgae biomass continuously in a location- and season-independent manner, i.e., throughout the year, with the same quality with respect to the composition of primary and secondary ingredients with a higher concentration of algae suspension in water per liter compared to the established processes (open pond, photobioreactor PBR and comparable technologies) for production of microalgae biomass.

An installation for production of microalgae biomass, which installation is proposed to achieve the object and is suitable for carrying out the process, comprises at least:
at least one cultivation module for phototrophic or mixotrophic cultivation of microalgae contained in a suspension with water, wherein said cultivation module consists of a gas portion and a liquid portion having a liquid reservoir,
inlets and application elements for introduction of CO₂, other nutrients and oxygen into the cultivation module,
at least one centrifuge for centrifugation of microalgae out of volume fractions of the suspension that are discharged from the at least one cultivation module (climatic chamber) for the purpose of harvesting,
a piping system having pumps for movement of the suspension between the aforementioned components and for supply of substances to be introduced, including nutrients and volume fractions of the suspension, into the cultivation module via the respective application elements,
a control device for control of the at least one centrifuge and of the aforementioned pumps and application elements in accordance with the results of the evaluation of sensor signals received thereby from sensors in the aforementioned components.

In this connection, the gas portion of the at least one cultivation module is equipped with at least one nozzle arranged in the upper region thereof for application of suspension, with at least one artificial light-emitting light source, preferably with multiple LED light source(s), and with structural elements for retardation of the gravitational downward movement of droplets that arise upon spraying the suspension. The light of the aforementioned at least one light source is tailored or tailorable to the species of the microalgae to be cultivated with regard to the wavelength and intensity thereof.

The basis of the distinction between a gas portion and a liquid portion of the at least one cultivation module is that the suspension of microalgae and water is actually present as a liquid in the proper sense in the portion of the cultivation module regarded as the liquid portion, whereas the gas portion contains the suspension as a finely atomized aerosol, owing to the atomization thereof that takes place by means of nozzles arranged in said gas portion, and CO₂ as gaseous nutrient. In this respect, the liquid portion of the cultivation module is formed by the liquid reservoir already mentioned (at the bottom of the gas portion or in a container below the gas portion) and by pipes of the piping system already addressed above, which pipes connect said liquid reservoir to the nozzle or the nozzles in the upper region of the gas portion, and having pumps and valves arranged therein and having optionally further components such as intermediate tanks and the like incorporated in the piping system. This understanding means that, in turn, not all parts of the piping system belong to the liquid portion or to the actual cultivation module, such as, for instance, pipe sections connecting the cultivation module to the centrifuge and the components arranged in said pipe sections.

It is very deliberate that reference is also made to a liquid reservoir and not to a suspension reservoir in connection with the liquid portion. The reason for this is that a cultivation module of the installation according to the invention, in connection with the first start-up thereof or its start-up following modification, is initially subjected to a so-called “water run”, in which pure water is initially circulated once or multiple times through the cultivation module using its liquid reservoir in the liquid portion that is still filled with water at this time and the water is only then inoculated with microalgae to form the suspension that is later stored by the liquid reservoir. It should be noted here that it is, however, also optionally possible to directly introduce a suspension containing already “precultivated” microalgae into the cultivation module or to inoculate initially introduced water by supply of suspension containing “precultivated” microalgae. The at least one cultivation module of the installation according to the invention is preferably of a size having a base area of at least 250 m².

According to the invention, the at least one cultivation module is designed as a climatic chamber which is operated in a water-economical manner in accordance with the process. It is self-evident that the temperature of the suspension conducted repeatedly through the cultivation module is regulated in a controlled manner by means of the control device. Whatever temperature is to be set for the suspension depends on the species of whichever microalgae are to be cultivated. It is clear that, for example, microalgae native to polar seas in line with their natural occurrence require distinctly lower temperatures for successful cultivation than, for example, microalgae species that occur naturally in waters of European regions having a moderate climate.

For water-economical operation of the at least one cultivation module, multiple sensors and application elements operatively connected to the control device are arranged both in the gas portion of said cultivation module and in the liquid portion of said cultivation module. Specifically, these are firstly, in particular, sensors (electrodes) arranged in the liquid portion for determination of the pH of the suspension, and application elements which are actuated for controlled addition of buffer ions in the reservoir by the control device according to whatever pH is established by means of the aforementioned sensors. Furthermore, the liquid portion of the cultivation module (climatic chamber) has arranged therein sensors for repeated measurement of the redox potential of the suspension, the sensor signals of which are processed by the control device for regulation of the redox potential by controlled supply of nutrients, including CO₂, and of oxygen and for control of the light intensity of the light emitted by the preferably multiple light sources (particularly preferably LED light sources).

The components required for water-economical operation of the climatic chamber (of the cultivation module climate-controlled in water-economical operation) are completed by sensors for repeated measurement of the redox potential of the suspension containing a very high proportion of water that is resupplied to the gas portion of the cultivation module and that remains upon centrifugation of the volume fractions discharged for harvesting, and also by a UV light source. The aforementioned sensors and the UV light source are preferably arranged in a pipeline for recycling of these volume fractions of the suspension into the cultivation module. As already stated, said sensors are used to repeatedly measure the redox potential of the suspension containing a high proportion of water and a few microalgae possibly still remaining therein that is to be recycled into the cultivation module after centrifugation, and the suspension to be recycled is irradiated with UV light in a controlled manner by means of the control device until a minimum redox potential is reached.

According to calculations which have been made, what can be generated by means of a cultivation module of the installation according to the invention on an area of about 250 m² is a quantity of microalgae biomass that would require a tube length of about 500 km for generation thereof by means of a tubular bioreactor. In connection with practical implementation of the proposed solution, it might be possible, for example, to regard tubular reactors of small to medium size as raw material suppliers, in that microalgae are in a sense “precultivated” therein and then supplied, as a constituent of a suspension, to a cultivation module of an installation designed according to the invention for the purpose of intensive cultivation under process conditions which are exactly adjustable, especially for the climatic chamber operated in a water-economical manner.

The structural elements arranged in the gas portion of a particular cultivation module for retardation of the downward movement of the microalgae-containing droplets can be, for example, horizontally arranged planar elements in the form of tautly stretched textile nonwovens or fine-meshed textile meshes. The structural elements in question are preferably very tautly stretched in order to avoid strong sagging thereof owing to wetting by the suspension and to thereby avoid creation of shaded regions within the gas portion of the cultivation module.

For particularly good and homogeneous flooding of the gas portion of the cultivation module with the light emitted by the at least one LED light source, preferably by the multiple LED light sources, the walls of the cultivation module in the gas portion are (optionally fully) mirrored, or high-gloss reflective on the their inner surface, in a particularly advantageous embodiment of the installation according to the invention. The latter means that the walls can be formed from a reflective high-gloss material or coated with such a material (optionally over the entire surface). This results in a particularly efficient utilization of the light energy introduced into the cultivation module.

In addition, the temperature of the suspension can advantageously be controlled using the waste heat of the LED light sources (the presence of multiple light sources is assumed in what follows). To this end, a cooling medium used for cooling of the LED light sources is appropriately regulated with respect to its volumetric flow rate, and so the surface temperature of each LED light source can be adjusted with the aid of the control device proceeding from the respective contribution of said LED light source to the heat input into the climatic chamber and according to whatever temperature is required for the suspension. Unlike with hitherto known greenhouses or installations for cultivation of microorganisms, the influence of heat introduced into such a system by light sources can be specifically controlled as a result.

This is not the case with hitherto known systems, since the heat quantity introduced by any artificial light sources is regularly neither known nor controllable with said systems. But on the other hand, it is by no means the case that, if a higher light intensity is required, a higher temperature is also absolutely necessary in the particular system (cultivation installation, greenhouse).

For the large-scale production of microalgae biomass, an installation designed according to the invention preferably comprises a plurality of cultivation modules and at least one centrifuge, but also optionally multiple centrifuges, shared by multiple cultivation modules for centrifugation of the biomass for the purpose of harvesting thereof. Such a modular structure has the advantage that if, for example, the microalgae cultivation process does not proceed as desired in one cultivation module or undesirable microbial contamination possibly occurs therein, only the suspension circulated within this one cultivation module has to be discarded, whereas microalgae having whatever are the required constitution and quality can continue to be harvested from the suspension of other cultivation modules of the installation. In contrast to all comparable systems competing for productivity, the cultivation module or a cultivation module of the installation according to the invention is a controlled, closed system. The risk of undesirable contamination, as in the case of open pond systems for example, can be virtually ruled out as a result.

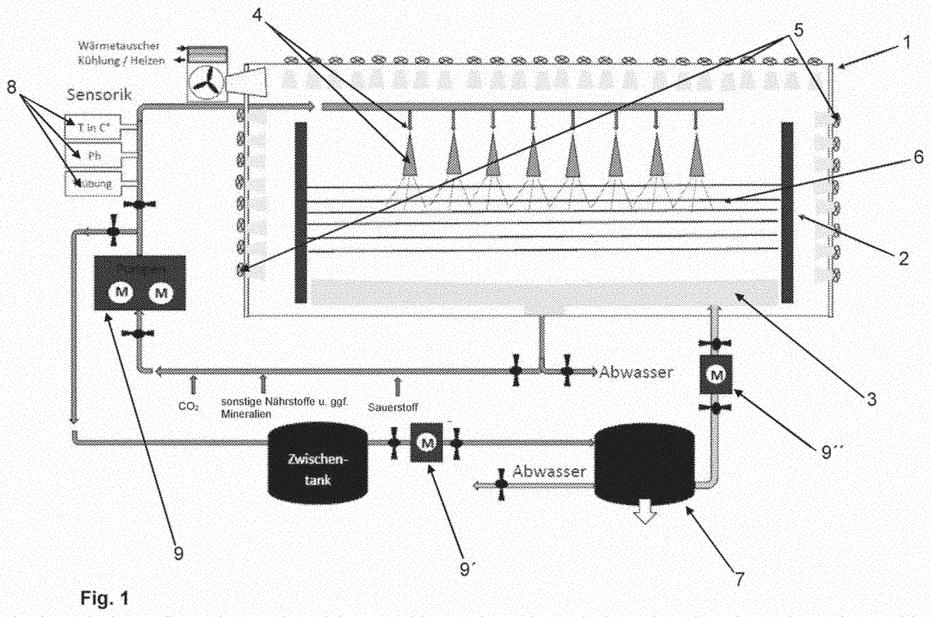

Some aspects of the invention shall be more particularly elucidated once again in what follows with reference to FIG. 1.

FIG. 1 shows, by way of example, a possible embodiment of a cultivation module of the installation according to the invention, including a centrifuge coupled thereto. The cultivation module forms a central component or the central component of the installation according to the invention for production of microalgae biomass, which component is ultimately also the focus of the invention, and so many other components of the installation (e.g., controller[s] and the like) are not depicted. Depending on the design of an installation configured according to the invention, it can comprise a relatively large number of such cultivation modules. In such a case, a plurality of said cultivation modules work together with one centrifuge (not depicted here) used for harvesting of the microalgae by centrifugation out of the suspension. In very large facilities, multiple centrifuges can optionally also be present, though typically not each cultivation module is separately assigned one centrifuge, but rather each of the centrifuges will work together with multiple cultivation modules.

The cultivation module 1 which is shown by way of example in FIG. 1 and which, as mentioned, may be present more than once in an installation designed according to the invention consists of the gas portion 2 and the liquid portion comprising a liquid reservoir 3 for accommodation of a supply of suspension consisting of water and the microalgae to be cultivated. As can be seen from FIG. 1, the liquid reservoir 3 is arranged at the bottom of the gas portion 2 of the cultivation module 1. In a process or cycle that is continuous apart from any changeover times, volume fractions of the suspension are fed from the liquid reservoir 3 to the gas portion 2 of the cultivation module 1 designed as a climatic chamber operated in a water-economical manner. This is done with the aid of the pump(s) 9 via pipes of a piping system, which pipes belong to the liquid portion of the cultivation module 1.

The suspension fed to the climatic chamber, i.e., to the cultivation module 1 climate-controlled in a water-economical mode of operation, is applied by spraying in the upper region of the gas portion 2—i.e., preferably directly below the ceiling—with the aid of multiple nozzles 4. Arranged in the gas portion 2 of the cultivation module 1 are multiple LED light sources 5—here, in the form of one or more light strips installed on the ceiling and the walls of the gas portion 2 of the cultivation module 1—which are controllable with regard to the wavelength and the intensity of the light emitted thereby. In addition, the cultivation module 1 has inlets for supply of nutrients, including CO₂ as gaseous nutrient, and oxygen for the microalgae cultivated therein.

The volume fractions of the suspension that are sprayed in the gas portion 2 of the cultivation module 1 via nozzles 4 form, as a result of the spraying, a mist (aerosol) of droplets each containing microalgae. The droplets gradually move to the bottom of the gas portion 2 of the cultivation module 1 while they are floating, the gas portion 2 also having arranged therein structures 6 which are formed by textile meshes or textile nonwovens and which retard the gravitational downward movement of the droplets. The purpose of this measure is to prolong the residence time of the volume fractions of the suspension sprayed in the cultivation module 1, i.e., the droplets containing the microalgae, in the gas portion, so that they are exposed to the artificial light of the LED light sources 5 and to the CO₂ introduced into the cultivation module 1 for as long as possible to promote algae growth and algae proliferation.

The suspension accumulating at the bottom of the gas portion 2 of the cultivation module 1 as a result of droplets admitted to the liquid reservoir 3 is refed from here to the gas portion 2 of the cultivation module 1. As the suspension circulates multiple times in this circuit, the turbidity of the suspension gradually increases owing to algae growth. The turbidity of the suspension repeatedly fed to the gas portion 2 of the cultivation module 1 is constantly determined by means of optical sensors 8 arranged in the pipelines. To this end, the installation according to the invention comprises a control device (not shown) operatively connected to the aforementioned sensors 8 and to further sensors.

The control device can be a central control unit or one due to multiple control units arranged in a decentralized manner and jointly forming the control device. Moreover, the control device is operatively connected to multiple actuators controlled thereby, such as application elements (these include the nozzles 4 in the gas portion 2 of the cultivation module 1) and controllable valves, in accordance with the results of the evaluation of sensor signals received from the sensors 8. If the turbidity of the volume fractions of the suspension repeatedly supplied to the gas portion 2 of the cultivation module 1 exceeds a minimum value defined in the control device by appropriate configuration, the control device causes a portion (volume fraction) of the suspension present in the liquid portion at that moment to be discharged from the cultivation module 1 and to be fed to the centrifuge 7.

In the centrifuge 7, the microalgae contained in the discharged volume fractions of the suspension are centrifuged out and supplied to subsequent processing operations which may no longer be carried out in the installation considered here. The suspension which remains upon centrifugation of the volume fractions discharged from the cultivation module 1 and which very predominantly consists of water is recycled back into the cultivation module 1, though it is treated beforehand by irradiation with UV light to eliminate microbial contamination. The latter takes place in a section of the pipe connection via which these volume fractions of the suspension predominantly consisting of water are resupplied to the cultivation module 1. In the pipe section equipped with an appropriate UV light source (not shown here), the residence time of the suspension remaining after centrifugation of the volume fractions discharged from the cultivation module 1 depends on the time required to kill any microbial contamination, with the suspension remaining in the region of UV light input until a minimum redox potential is reached, which redox potential is established by means of sensors (electrodes) (likewise not shown) within the pipe section between the output of the centrifuge 7 and the cultivation module 1.

To realize the water-economical mode of operation of the climatic chamber forming the cultivation module 1, yet further sensors 8 are arranged at least in the liquid portion of said climatic chamber, which sensors 8 are operatively connected to the control device (not shown). These are at least sensors 8—for example in the form of silver chloride electrodes—for determination of the pH of the suspension and sensors 8—likewise specific electrodes—for determination of the redox potential of the suspension. It is in accordance with the result from the continuous measurement of the pH of the suspension that the control device controls a metered addition of buffer ions, namely calcium ions and/or magnesium ions, by actuation of corresponding application elements (likewise not shown here in detail) arranged for this purpose in the liquid portion of the cultivation module 1.

The redox potential of the suspension is regulated in a controlled manner by means of the control device, in that, in the event of the redox potential falling below 100 mV, the supply of nutrients (nutrients introduced into the liquid portion and the gaseous nutrient CO₂ ultimately applied in the gas portion 2 of the cultivation module 1) is stopped and the intensity of the light emitted by the LED light sources 5 in the gas portion 2 of the cultivation module 1 is reduced, with the input of oxygen being increased at the same time by actuation of corresponding application elements in the liquid zone. If the event of an excessively high redox potential, namely a redox potential of more than 300 mV, the light intensity in the gas portion 2 of the cultivation module 1 is increased and the input of nutrients, namely the CO₂ introduced into the cultivation module 1 and other nutrients introduced, is increased.

In order to bring about a substantially constant input of energy throughout the cultivation cycle in the form of the light emitted by the LED light sources 5, the light intensity in the gas portion 2 of the cultivation module 1 is also increased as the turbidity of the suspension increases, which turbidity is established by sensor in the liquid portion of the cultivation module 1. To support the temperature control of the suspension, the controller, on the basis of the temperature of the suspension ascertained by means of at least one temperature sensor in the liquid portion of the cultivation module 1, can control the volumetric flow rate of a cooling medium conducted through active cooling elements for the LED light sources 5 and thereby control the surface temperature of the LED light sources 5 emitting not only light but also heat (as a by-product in a sense) into the cultivation module 1, it optionally also being possible for the temperature control of the suspension to be effected exclusively on the basis of such control.

1. A process for producing microalgae biomass, in which microalgae contained in a suspension of water and microalgae are phototrophically or mixotrophically cultivated in a continuous circulation in a cultivation module through which the suspension passes multiple times and which comprises a gas portion and a liquid portion having a liquid reservoir with supply of light from at least one artificial light source, which light evenly floods the entire gas portion of the cultivation module, and of nutrients, wherein volume fractions of the suspension are repeatedly discharged from the cultivation module for harvesting of microalgae and centrifuged by means of a centrifuge and the suspension remaining after centrifugation is supplied back to the cultivation module, characterized in that volume fractions of the suspension are discharged for harvesting of microalgae whenever the turbidity of the suspension, as established by means of optical sensors, exceeds a minimum value, and in that the microalgae are cultivated in a climatic chamber which forms the cultivation module and which is operated in a water-economical manner by not only regulating the temperature of the suspension, but also regulating the pH thereof by means of controlled addition of buffer ions and regulating the redox potential of the suspension and hence the microbial contamination thereof by means of control of the supply of light and nutrients and of metered addition of oxygen, and by irradiating the suspension remaining after centrifuging out the microalgae, before it is recycled into the cultivation module, with UV light to kill undesirable microbial contamination until a minimum redox potential measured in the suspension is reached.

2. The process as claimed in claim 1, characterized by the process steps which are passed through multiple times:
a) spraying volume fractions of the suspension, which volume fractions contain microalgae to be cultivated and are taken from the liquid reservoir of the liquid portion, in the upper region of the gas portion of the cultivation module into which nutrients, including at least CO₂ as gaseous nutrient, are introduced,
b) retarding the gravitational downward movement of droplets of the suspension that arise upon spraying and contain the microalgae, by means of suitable structural elements arranged for this purpose in the gas portion of the cultivation module,
c) exposing the microalgae contained in the droplets retarded in their downward movement to the light from LED light sources arranged in the gas portion of the cultivation module, which light is tailored to the species of the microalgae to be cultivated with respect to the wavelength thereof and the intensity thereof, and to the introduced CO₂,
d) recycling downwardly moving, microalgae-containing droplets into the liquid reservoir formed at the bottom of or in a container below the gas portion of the cultivation module after passage through the gas portion of the cultivation module,
e) discharging volume fractions of suspension recycled for respraying in the gas portion of the cultivation module from the liquid reservoir and harvesting microalgae contained in the discharged volume fractions by centrifugation in a centrifuge if the turbidity of the volume fractions of the suspension that are supplied back to the gas portion of the cultivation module for respraying as per process step a), which turbidity is established by means of optical sensors, exceeds a minimum value, and also recycling the suspension remaining after centrifugation of the discharged volume fractions into the cultivation module after irradiation with UV light,
wherein
the pH of the suspension in the liquid portion is measured and is regulated by controlled metered addition of buffer ions and is thereby kept within a pH range between 7 and 8, preferably between 7 and 7.5, that promotes the growth of the microalgae,
the microbial contamination of the suspension is regulated through monitoring of the redox potential thereof by increasing the supply of nutrients and the light intensity of the LED light sources if the redox potential exceeds an upper limit and reducing the supply of nutrients and the light intensity of the LED light sources and also increasing the metered addition of oxygen that takes place in the liquid portion of the cultivation module if the redox potential falls below a lower limit, wherein the light intensity of the LED light sources is additionally controlled depending on the turbidity of the suspension, which turbidity is determined as per process step e), such that it is adjusted proportionally to the turbidity.

3. The process as claimed in claim 2, characterized in that the surface temperature of the LED light sources arranged in the gas portion of the cultivation module is used for temperature control of the suspension, which surface temperature is regulated by reducing or increasing the volumetric flow rate of a cooling medium used for cooling of the LED light sources.

4. The process as claimed in claim 1, characterized in that calcium ions and/or magnesium ions are metered in as buffer ions for the carbonic acid equilibrium for regulation of the pH of the suspension in the liquid portion of the cultivation module.

5. The process as claimed in claim 1, characterized in that nitrogen, phosphorus and carbon are supplied as nutrients to the suspension in the liquid portion of the cultivation module.

6. The process as claimed in claim 1, characterized in that secondary ingredients are formed or enriched in the microalgae by supplying in a controlled quantity to the suspension in the liquid portion of the cultivation module substances or groups of substances that belong to at least one of the following categories:
nutrients and microbiological contamination for formation of vitamins,
nutrients for formation of omega-3 fatty acids,
nutrients for formation of bioactive proteins/peptides,
minerals to be bound by the microalgae,
zinc or iron to be bound by the microalgae.

7. The process as claimed in claim 1, characterized in that volume fractions of the suspension in the order of magnitude of 15% to 50% of the volume of suspension present in the liquid portion of the cultivation module are discharged from the cultivation module if the minimum value defined for the turbidity of the suspension is exceeded.

8. An installation for production of microalgae biomass, wherein the installation comprises at least
at least one cultivation module for phototrophic or mixotrophic cultivation of microalgae contained in a suspension with water, consisting of a gas portion and a liquid portion having a liquid reservoir and having at least one nozzle arranged in an upper region of the gas portion as application element for suspension, having at least one light source for emission of artificial light tailored to the species of the microalgae to be cultivated with respect to the wavelength thereof and the intensity thereof, which light source is arranged in the gas portion, and also having structural elements arranged in the gas portion for retardation of the gravitational downward movement of droplets that arise upon spraying the suspension,
inlets and application elements for introduction of CO₂, other nutrients and oxygen into the at least one cultivation module,
at least one centrifuge for centrifugation of microalgae out of volume fractions of the suspension that are discharged from the at least one cultivation module and supplied to the centrifuge for the purpose of harvesting of the microalgae,
a piping system having pumps for movement of the suspension between the aforementioned components and for supply of substances to be introduced, including nutrients and volume fractions of the suspension, into the cultivation module via the respective application elements,
a control device for control of the at least one centrifuge and of the aforementioned pumps and application elements in accordance with the results of the evaluation of different sensor signals received from sensors which are arranged in the components of the installation and are operatively connected to the controller,
characterized in that
the at least one cultivation module is designed as a climatic chamber which is operated in a water-economical manner by not only regulating the temperature of the suspension by means of the control device on the basis of sensor signals of the sensors arranged in the at least one cultivation module, but also regulating the pH of the suspension by means of controlled addition of buffer ions into the liquid portion of the at least one cultivation module and regulating the redox potential of the suspension, which redox potential is measured repeatedly by means of sensors, by means of control device-controlled supply of the CO₂, the other nutrients and oxygen into the at least one cultivation module and a control of the light intensity in the gas portion of the at least one cultivation module, and by irradiating suspension which remains upon centrifugation of the microalgae out of discharged volume fractions and which is resupplied to the at least one cultivation module, before it is reintroduced into the at least one cultivation module, with UV light in a controlled manner by means of the control device to kill undesirable microbial contamination until a minimum redox potential measured by sensor in the suspension to be recycled is reached.

9. The installation as claimed in claim 8, characterized in that the gas portion of the at least one cultivation module has reflective walls which are mirrored or high-gloss reflective on their inner surface.

10. The installation as claimed in claim 9, characterized in that the side walls and the ceiling of the gas portion of the at least one cultivation module are fully mirrored or fully high-gloss reflective on their inner surface.

11. The installation as claimed in claim 8, characterized in that the at least one light source arranged in the gas portion of the at least one cultivation module simultaneously acts as heating for temperature control of the suspension in said at least one cultivation module, wherein the waste heat caused by the surface temperature of said at least one light source is controlled by the control device by means of control of the volumetric flow rate of a liquid cooling medium used for active cooling of the at least one light source.

12. The installation as claimed in claim 8, characterized in that the at least one cultivation module has arranged on the side walls or on the side walls and the ceiling of the gas portion thereof one or more light strips composed of LEDs as light sources.

13. The installation as claimed in claim 8, characterized in that the structural elements for retardation of the downward movement of the droplets formed by spraying of the suspension, which structural elements are arranged in the gas portion of the at least one cultivation module, are horizontally arranged, tautly stretched planar elements composed of a textile nonwoven or of a textile fine-meshed mesh.

14. The installation as claimed in claim 8, characterized in that the installation comprises a plurality of cultivation modules which each comprise a gas portion and a liquid portion having a liquid reservoir, wherein a centrifuge for centrifugation of volume fractions of the suspension discharged for harvesting of microalgae is jointly assigned to multiple or all cultivation modules.

15. The installation as claimed in claim 14, characterized in that it comprises up to 40 cultivation modules having a base area of at least 250 m² in each case.
